(19) United States
(12) Patent Application Publication
(10) Pub. No.: US 2004/0214992 A1
(43) Pub. Date: Oct. 28, 2004

(54) Mutation induced optimization of receptor signal to noise ratio

(76) Inventors: Alan S. Kopin, Wellesley, MA (US); Martin Beinborn, Boston, MA (US)

Correspondence Address: CLARK & ELBING LLP, 101 FEDERAL STREET, BOSTON, MA, 02110, US

(21) Appl. No.: 10/138,126

(22) Filed: May 3, 2002

Related U.S. Application Data
(60) Provisional application No. 60/288,644, filed on May 3, 2001.

(51) Int. Cl. C07K 14/72; C07H 21/04

(52) U.S. Cl. 530/358; 536/23.5; 435/69.1; 435/320.1; 435/325

(57) ABSTRACT

The present invention provides an alternative strategy for optimizing the signal to noise ratio of a given receptor. Specifically, the present invention provides receptor mutants having an increased signal to noise ratio. In one preferred embodiment, the present invention provides receptor mutants having a decreased level of basal activity. As part of this aspect, the present invention provides a mutant serotonin receptor and a mutant CCR-3 receptor, each having decreased basal activity. In another preferred embodiment, the present invention provides receptor mutants having an increased maximal level of ligand induced signaling. Such receptors optimize the signal to noise ratio of a receptor and provide, for example, for more sensitive screens for drug discovery.

This application claims the benefit of the filing date of U.S. provisional application, U.S.S.No. 60/288,644, filed May 3, 2001.

### BACKGROUND OF THE INVENTION

In general, the invention features receptors having an optimized signal to noise ratio that are useful in ligand screening assays.

Optimization of the signal to noise ratio in an assay is a critical element in detecting the activity being measured. This applies particularly to assays for detecting the activity of receptors, for example, G protein-coupled receptors, single transmembrane receptors, and nuclear receptors. The activity of a particular receptor (e.g., the ligand binding or signaling activity) is typically measured using an assay that detects the intracellular signal transduced by the particular receptor in response to ligand binding.

A wide variety of assays are available in the art for measuring receptor activity. For example, in one commonly used assay, cells of a selected type are transfected in vitro with DNA encoding a particular receptor and the basal and/or ligand-stimulated receptor activity is measured. Typically, the ligand-stimulated receptor activity measured is the induction of an intracellular second messenger signal. Alternatively, the transcriptional activation of a particular gene, which is known to be activated by the biochemical pathway induced by the receptor, can serve as a transcriptional readout for receptor activity using a standard reporter assay.

Prior to the present invention, receptor assays have been optimized by a variety of approaches, such as by altering the amount of DNA transfected into the cells, altering the cells used for transfection, changing the time course of signaling (e.g., by altering the time period over which the cells are allowed to grow post-transfection or altering the period of time the cells are contacted by the ligand for the receptor), examining different second messengers, or examining second messenger induced transcriptional readouts (e.g., by using a transcriptional reporter assay). Such approaches require testing a variety of parameters with no assurance of actually optimizing, or even improving, the signal to noise ratio of the assay.

### SUMMARY OF THE INVENTION

The present invention provides a method of optimizing assays for receptor activity that reliably reduces the signal to noise ratio of a particular receptor, and also provides mutant receptors having an optimized signal to noise ratio. Optimization of the signal to noise ratio of an assay for receptor activity is achieved by: (1) decreasing the basal level of signaling of the receptor (silenced receptor), or (2) increasing the level of ligand-stimulated second messenger signaling of the receptor (activated receptor).

In a first aspect, the invention features a silenced receptor having an increased signal to noise ratio compared to a corresponding wild-type receptor, the silenced receptor having decreased basal activity compared to the wild type receptor. In embodiments of this aspect, the silenced receptor is a G protein-coupled receptor, such as a serotonin receptor (e.g., a serotonin 2A receptor, which may have a Lys to Glu mutation at position 323 of SEQ ID NO: 1) or a CCR-3 receptor (e.g., a CCR-3 receptor having a Tyr to Glu mutation at position 235 of SEQ ID NO: 2). In other embodiments of this aspect, the silenced receptor is a nuclear receptor, a steroid hormone receptor, or a single transmembrane receptor.

In a second aspect, the invention provides an activated receptor having an increased signal to noise ratio compared to a corresponding wild-type receptor, the activated receptor having increased ligand-stimulated activity compared to the wild-type receptor. In embodiments of this aspect, the activated receptor is a G protein-coupled receptor, a nuclear receptor, or a single transmembrane receptor.

In a third aspect, the invention provides a kit including a silenced receptor having an increased signal to noise ratio compared to a corresponding wild-type receptor, the receptor having a decreased basal activity compared to the wild-type receptor. In embodiments of this aspect, the silenced receptor is a G protein-coupled receptor, such as a serotonin receptor (e.g., a serotonin 2A receptor, which may have a Lys to Glu mutation at position 323 of SEQ ID NO: 1) or a CCR-3 receptor (e.g., a CCR-3 receptor having a Tyr to Glu mutation at position 235 of SEQ ID NO: 2). In other embodiments of this aspect, the silenced receptor is a nuclear receptor, a steroid hormone receptor, or a single transmembrane receptor.

In a fourth aspect, the invention provides a kit including an activated receptor having an increased signal to noise ratio compared to a corresponding wild-type receptor, the activated receptor having an increased ligand-stimulated activity compared to the wild-type receptor. In embodiments of this aspect, the activated receptor is a G protein-coupled receptor, a nuclear receptor, or a single transmembrane receptor.

In a fifth aspect, the invention provides a method of using a receptor having an increased signal to noise ratio to identify ligands for the receptor by cotransfecting cells with an expression vector containing a nucleic acid encoding the receptor having an increased signal noise ratio and a receptor activation-sensitive reporter construct, the reporter construct including an operably linked response element, which is sensitive to activation by the receptor, promoter, and reporter gene; contacting the cells with a candidate ligand; and assaying for alterations in the basal or ligand-stimulated activity of the reporter construct, an increase or decrease in the ligand-dependent activation of the receptor, compared to ligand-independent signaling, indicating the presence of an agonist or antagonist, respectively. In embodiments of this aspect, the receptor having increased signal to noise ratio is a silenced receptor or an activated receptor, either of which may be a G protein-coupled receptor, a nuclear receptor, or a single transmembrane receptor. The silenced G protein-coupled receptor may be a serotonin receptor (e.g., a serotonin 2A receptor, such as one having a Lys to Glu mutation at position 323 of SEQ ID NO: 1) or a CCR-3 receptor (e.g., a CCR-3 receptor having a Tyr to Glu mutation at position 235 of SEQ ID NO: 2).

“Basal” activity means the level of activity (e.g., activation of a specific biochemical pathway or second messenger signaling event) of a receptor in the absence of stimulation with a receptor-specific ligand (e.g., a positive agonist). The basal activity can be less than the level of ligand-stimulated activity of a wild-type receptor. However, in certain cases, a receptor with increased basal activity may display a level of signaling that approximates, is equal to, or exceeds the level of ligand-stimulated activity of the corresponding wild type receptor.

By “silencing mutation” or “silenced receptor” is meant a mutation that decreases the basal activity of a receptor to a level below the basal activity of the corresponding wild-type receptor or other negative control (e.g., a vector lacking a nucleic acid sequence encoding a receptor polypeptide). According to the present invention, a silencing mutation does not result in a reduction in ligand induced signaling of the receptor.

By “activated receptor” is meant a receptor having an increase in ligand-stimulated activity. The increase in ligand-stimulated activity may be due to increased expression levels as a result of a mutation.

By “negative control” is meant any construct that can be used to distinguish increases or decreases in the signaling of a candidate receptor. The appropriate negative control for any given candidate receptor will vary depending on the assay and the type of alterations in signaling. For example, for a silenced receptor, the appropriate negative controls may be a vector including wild type receptor nucleotide sequences, or a vector including constitutively active receptor nucleotide sequences. The appropriate negative control to be used to identify a silenced or activated receptors will be apparent to one of ordinary skill in the art.

By “signal to noise ratio” is meant the net quantitative difference between the measurable activity of a receptor in the absence of ligand stimulation and the measurable activity of a receptor in the presence of ligand stimulation.

By “disease” or “disorder” is meant any ailment or adverse condition that can be diagnosed in a mammal. As used herein, disease or disorder can be used to refer to a physical symptom such as a pain or an ache (e.g., chronic back pain or arthritis etc.) or to refer to a severe condition, such as cancer.

As used herein, “second messenger signaling activity” refers to the production of an intracellular stimulus (including, but not limited to, cAMP, cGMP, ppgpp, inositol phosphate, or calcium ions) in response to activation of the receptor, or to activation of a protein in response to receptor activation, including but not limited to a kinase, a phosphatase, or to activation or inhibition of a membrane channel.

A “naturally-occurring” receptor refers to a form or sequence of a receptor as it exists in an animal. Those skilled in the art will understand “wild type” receptor to refer to the conventionally accepted “wild-type” amino acid consensus sequence of the receptor, or to a “naturally-occurring” receptor with normal physiological patterns of ligand binding and signaling.

By a “corresponding wild-type receptor” is meant the “wild-type” or “naturally occurring” form of a mutant receptor that is silenced or activated.

A “mutant receptor” is understood to be a form of a receptor in which one or more amino acid residues in the predominant receptor occurring in nature (e.g., a naturally-occurring wild-type receptor) have been either deleted or replaced. Alternatively additional amino acid residues have been inserted.

By “substantially pure and isolated nucleic acid” is meant nucleic acid (e.g., DNA or RNA) that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

“Transformed cell” means a cell into which (or into an ancestor of which) has been introduced, by means of recombinant DNA techniques, a DNA molecule encoding (as used herein) a polypeptide described herein (for example, a CCR-3 receptor polypeptide).

“Promoter” means a minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the native gene. A promoter element may be positioned for expression if it is positioned adjacent to a DNA sequence so it can direct transcription of the sequence.

“Operably linked” means that a gene and a regulatory sequence(s) are connected in such a way as to permit gene expression when the appropriate molecules (e.g., transcriptional activator proteins) are bound to the regulatory sequence(s).

“Reporter assay system” means any combination of vectors typically used for measuring transcriptional activation. A typical reporter assay system includes at least a reporter construct and an expression vector encoding the polypeptide that activates (e.g., directly) or causes to activate (e.g., indirectly) expression of the reporter construct. The reporter assay system may also include additional expression vectors encoding other polypeptides that participate in activation of the reporter construct.

“Expression vectors” contain at least a promoter operably linked to the gene to be expressed.

A “reporter construct” includes at least a promoter operably linked to a reporter gene. Such reporter genes may be used in any assay for measuring transcription or translation and may be detected directly (e.g., by visual inspection) or indirectly (e.g., by binding of an antibody to the reporter gene product or by reporter product-mediated induction of a second gene product). Examples of standard reporter genes include genes encoding the luciferase, green fluorescent protein, or chloramphenicol acetyl transferase gene polypeptides (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Expression of the reporter gene is detectable by use of an assay that directly or indirectly measures the level or activity of the reporter gene. Preferred reporter constructs also include a response element.

A “response element” is a nucleic acid sequence that is sensitive to a particular signaling pathway, e.g., a second messenger signaling pathway, and assists in driving transcription of the reporter gene in cooperation with the promoter. As used herein, “response element” may also refer to a promoter that is activated in response to signaling through a particular receptor.

### DETAILED DESCRIPTION OF THE DRAWINGS

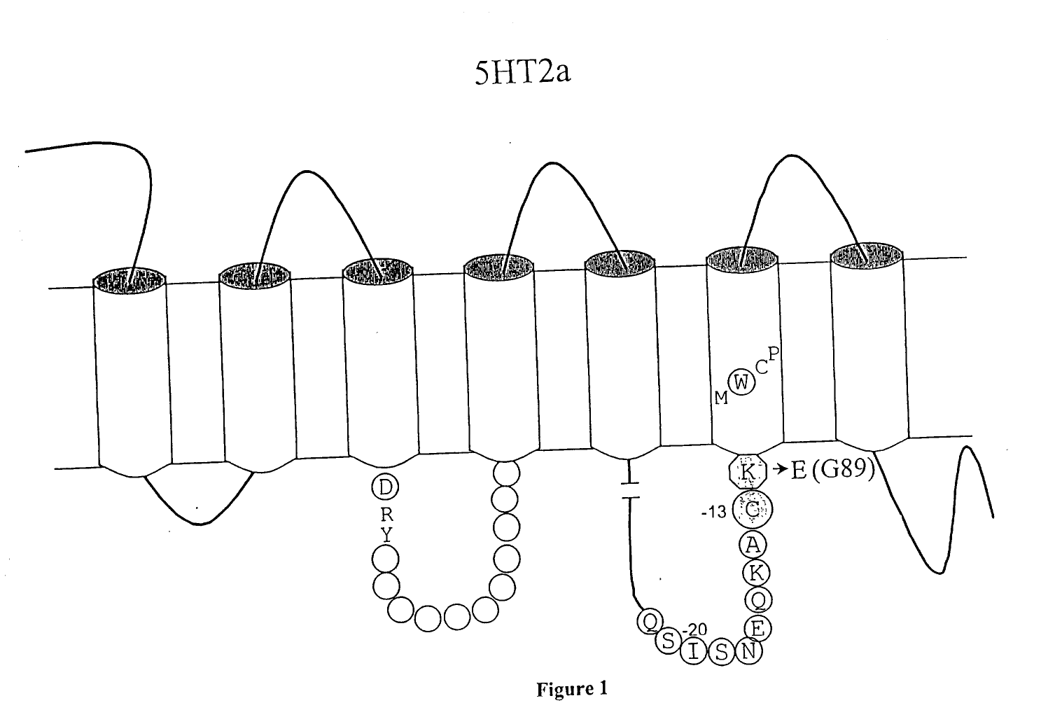

FIG. 1 is a schematic illustration of the human serotonin 2A receptor, showing the “DRY” motif and the residues at positions -13 and -20 relative to the “CWLP” motif, which are conserved between the rat and human serotonin 2A receptors. The illustration also indicates the particular mutation of the Lys residue at position 323 of SEQ ID NO: 1 to a Glu, which induces reduced basal signaling of the serotonin 2A receptor.

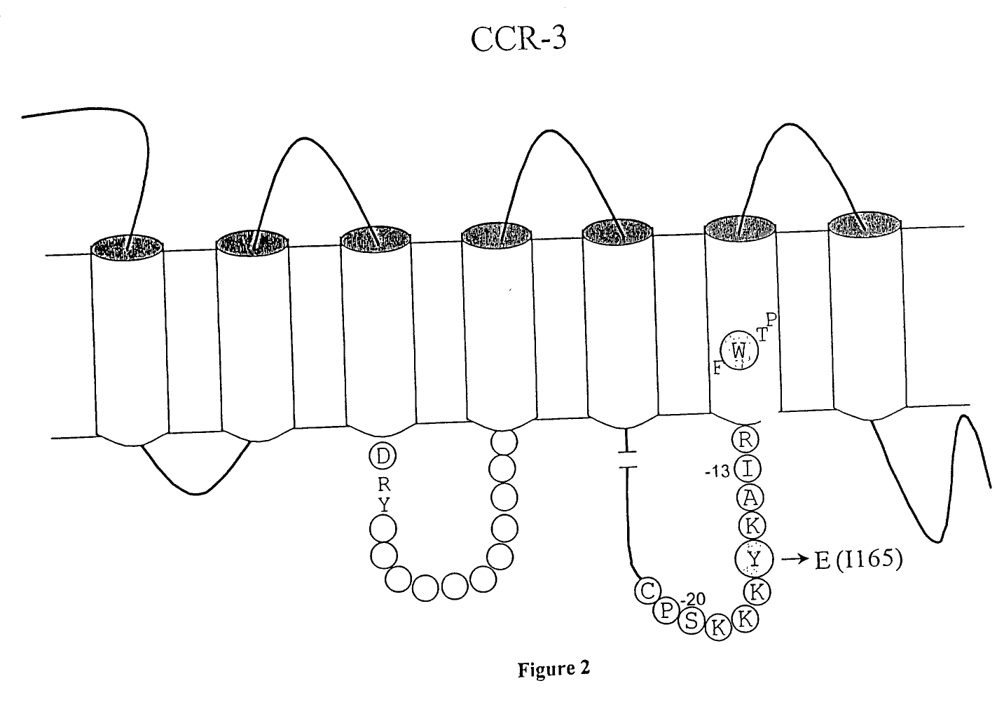

FIG. 2 is a schematic illustration of the human CC chemokine 3 (CCR-3) receptor, showing the “DRY” motif and the residues at positions -13 and -20 relative to the “CWLP” motif. In addition, the illustration indicates the mutation of the Tyr residue at position 235 of SEQ ID NO: 2 to a Glu, which induces reduced basal signaling of the CCR-3 receptor.

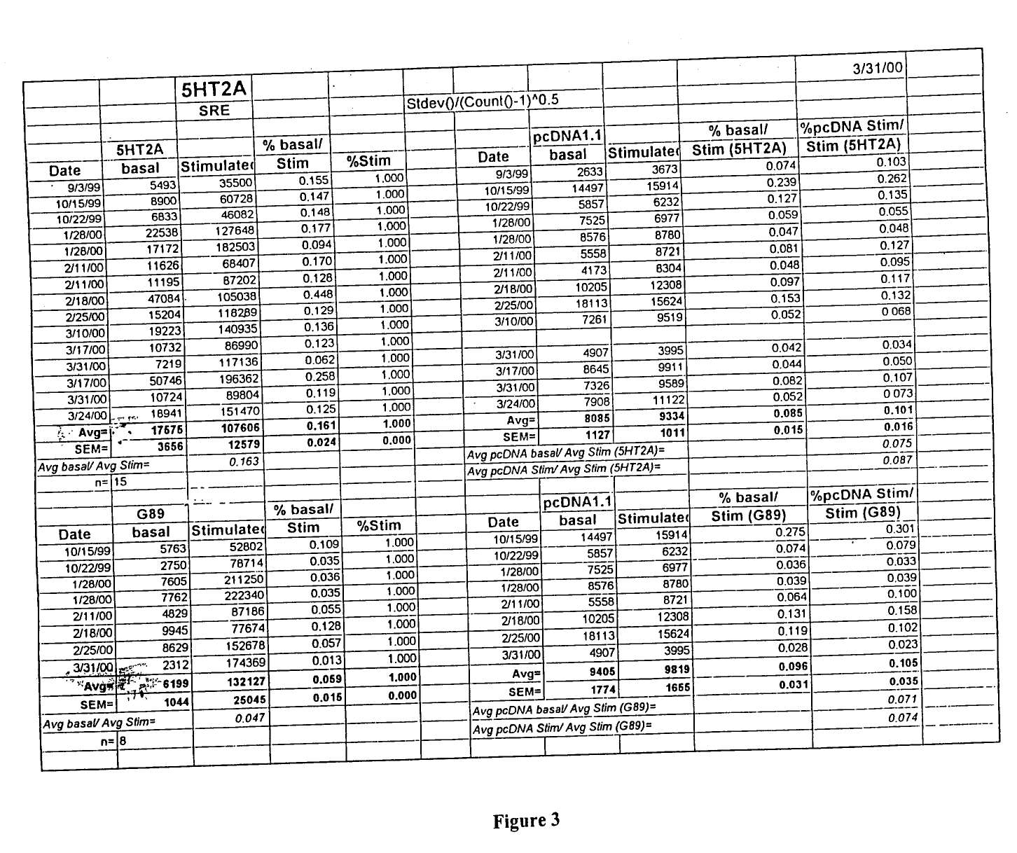

FIG. 3 is a table of experimental results measuring serotonin receptor stimulation by assaying for luciferase activity.

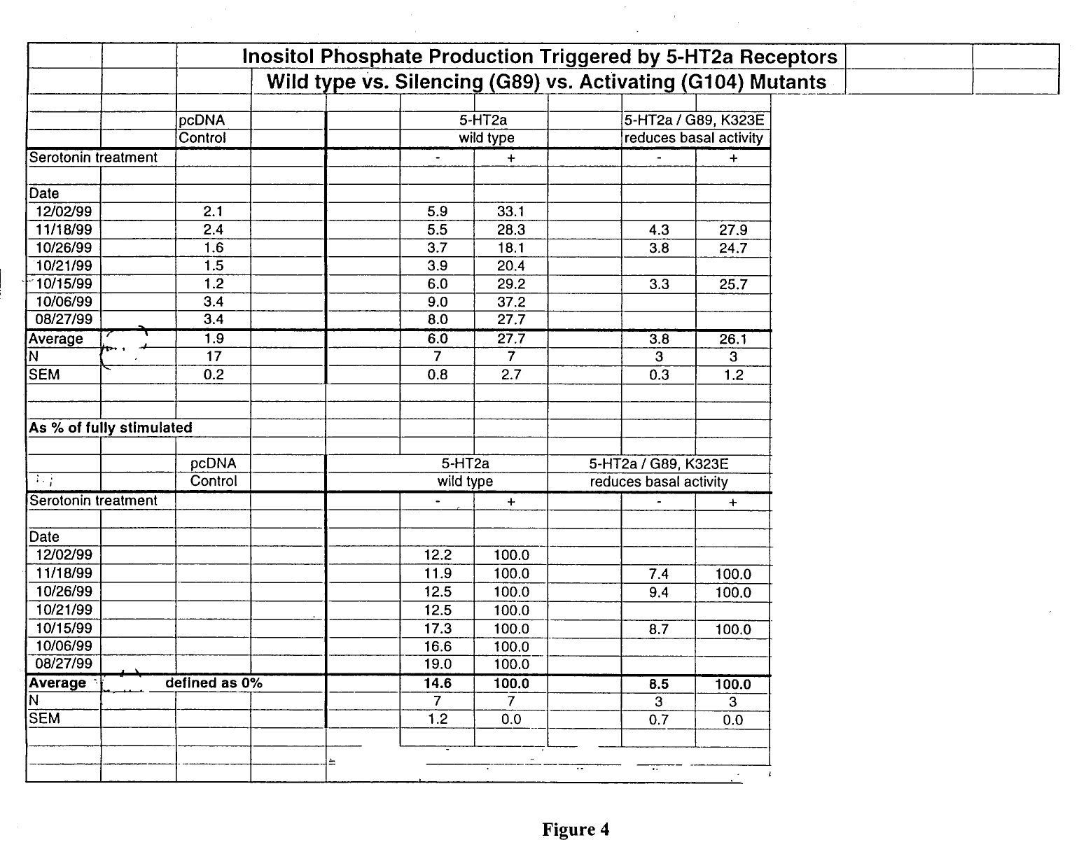

FIG. 4 is a table of experimental results measuring serotonin receptor stimulation by assaying for inositol phosphate production.

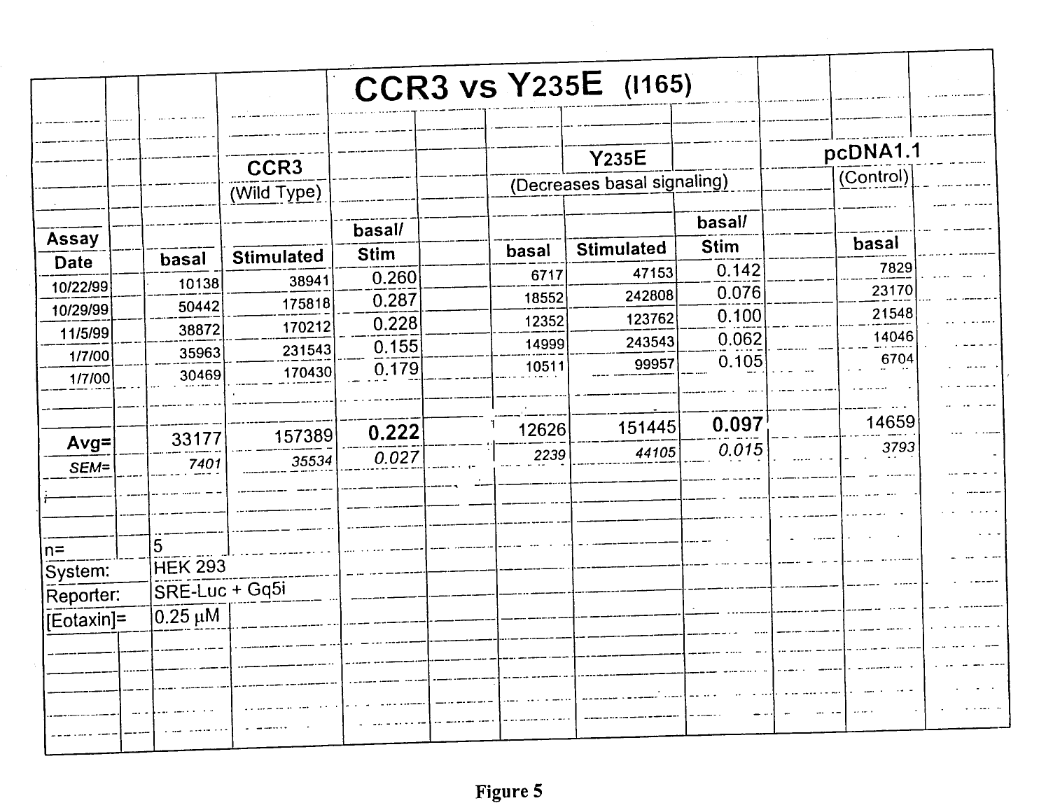

FIG. 5 is a table of experimental results measuring CCR-3 receptor stimulation by assaying for luciferase activity.

### DETAILED DESCRIPTION OF THE INVENTION

A major focus of current scientific research is the identification of novel receptor ligands. In this respect, receptors are valuable tools for use in large scale screening assays aimed at the identification of novel receptor binding ligands to identify agonist and antagonist drugs of a particular receptor. In order to maximize the detection potential of a particular ligand binding assay, it is advantageous to optimize the signal to noise ratio of the assay.

The present invention provides an alternative strategy for optimizing the signal to noise ratio of a given assay by altering the signaling of a given receptor. Specifically, the present invention provides receptor mutants having intrinsic increases in the signal to noise differential. In one embodiment, the present invention provides receptor mutants having decreased levels of basal activity. In another embodiment, the present invention provides receptor mutants having increased maximal levels of ligand-stimulated signaling. Both types of receptors are functionally abnormal receptors, compared to the corresponding wild-type receptors. In addition, both types of receptors serve as better screens for agonist or antagonist drugs by maximizing the net difference between basal and ligand induced activity. Such receptors optimize the signal to noise ratio of the receptor assay and provide a more sensitive screen for drug discovery.

For example, a decrease in the basal activity of a receptor effectively lowers the threshold for receptor activation, allowing the detection of agonist activity that might not otherwise be identified using the corresponding wild-type receptor. Similarly, an increase in the ligand induced signaling of a receptor amplifies the output signal of a receptor, allowing the identification of ligands exhibiting antagonist activity that might not otherwise be identified using the corresponding wild-type receptor. However, one skilled in the art will recognize that either type of receptor, e.g., a receptor with a decreased basal activity or a receptor with an increased ligand-stimulated activity, may be used to identify agonists or antagonists. Furthermore, one skilled in the art will appreciate that the inventive approach can be used with any receptor, including all classes of G protein-coupled receptors (including biogenic amine and orphan receptors), single transmembrane domain receptors, and nuclear receptors (e.g., steroid hormone receptors).

As noted above, the receptors of the present invention can be used in high-throughput drug screening assays to identify ligands (e.g., including peptide, non-peptide, and small molecule ligands) that bind to and activate the corresponding wild-type receptor. Identifying ligands that are agonists or antagonists of a receptor of therapeutic interest is the first step in narrowing the pool of candidate ligands that are targeted for further analysis. The goal of such an analysis is to identify and characterize useful therapeutic agents for treatment of a disease or disorder. Indeed, receptors having altered signaling, such as the silenced or activated receptors of the invention, are important tools for drug discovery due to the fact that a considerable number of diseases and other adverse effects can result from abnormal receptor activity. Thus, using the silenced or activated receptors of the invention, ligands for wild-type receptors of therapeutic interest may be identified that are key therapeutic agents.

The Serotonin Receptor

The serotonin 2A receptor (also referred to as the 5-hydroxytryptamine_{2A} (5HT2A) receptor) is a G protein-coupled receptor expressed primarily in areas of the brain, including the perirhinal cortex, the piriform cortex, the prefrontal cortex, the medial anterodorsal amygdala, and the CA 2-3 region of the hippocampus, as well as on human platelets and blood vessels (Serres et al., Eur Psychiatry 14(8):451-457 (1999); Kato et al., Mol. Cell. Biochem. 199(1-2):57-61 (1999); Osterlund et al., Brain Res Mol Brain Res 74(1-2):158-166 (1999); Hernandez et al., J. Neurosci. Res. 59(2):218-225 (2000)). Abnormalities in serotonin signaling, regulated by the serotonin receptor, specifically the serotonin 2A receptor, have been implicated in the pathophysiology of depressive disorders, for example, depression, bipolar affective disorder, mood disorders, anxiety, and schizophrenia (Massat et al., Am J. Med. Genet. 96(2):136-140; Serretti et al., J. Psychiat. Res. 34(2):89-98 (2000); Bromidge et al., J. Med. Chem. 43(6): 1123-1134 (2000); Serretti et al., Am J. Med. Genetic. 96(l):84-87 (2000); Serres et al., supra). For example, research has shown that the number of serotonin 2A receptors expressed in a depressed patient is increased (Osterlund et al., supra). In addition, a polymorphism in the serotonin 2A receptor gene has been identified in patients with anorexia nervosa and bulimia nervosa, which are also classified as depressive disorders (Necamias et al., Neurosci Lett 277(2):134-136 (1999).

The present invention provides a mutant serotonin 2A receptor that has reduced basal activity compared to the wild-type serotonin 2A receptor. This receptor classifies as a silenced receptor. Specifically, the silenced serotonin 2A receptor has a Lys to Glu substitution at amino acid 323 of SEQ ID NO: 1. This mutation was identified based on the high degree of conservation of the Cys residue at amino acid 322 of SEQ ID NO: 1 (indicated as position −13 relative to the “CWLP” motif in FIG. 1) between the human serotonin 2A receptor and the rat serotonin 2A receptor (see Pauwels et al., Biochem. 343 2:435-42 (1999); Egan et al., J. Pharm. Exp. Therap. 286(1):85-90, (1998)). In addition, it was known that substitutions at the -13 position in other receptors, particularly substitutions that resulted in a change in amino acid charge, frequently yielded receptors having an increase in basal activity (i.e., constitutive activity). Amino acid substitutions that altered the charge of amino acid 322 and surrounding residues were therefore tested to determine whether any of these substitutions yielded mutant receptors having altered basal activity. These mutant receptors were tested using a transcriptional reporter assay capable of measuring the basal activity of the serotonin 2A receptor. Cells were cotransfected with a luciferase reporter construct containing a serotonin response element (SRE) and one of either the wild-type serotonin 2A receptor, the mutant serotonin 2A receptor, or negative control pcDNA1. As shown in Example 1 below, the Lys323Glu serotonin receptor consistently yielded a decrease in receptor induced basal transcriptional activity.

The CCR-3 Receptor

The CC chemokine receptor 3 (CCR-3) receptor is a seven transmembrane G protein-coupled receptor expressed on thymocytes that plays a major role in the recruitment of inflammatory cells in an allergic response. Specifically, the CCR-3 receptor binds the polypeptide eotaxin to effect the regulation of eosinophil trafficking. The CCR-3 receptor also serves as a coreceptor for entry of the human immunodeficiency virus into cells. (See Franz-Bacon et al., Blood 93(10):3233-3240 (1999); Zimmermann et al., J. Immunol. 164(2):1055-1064 (2000); Zimmermann et al., Biochim. Biophys Acta 1442(2-3):170-176 (1998).)

The present invention provides a mutant CCR-3 receptor having reduced basal activity compared to the corresponding wild-type receptor. This silenced CCR-3 receptor has a substitution of the Tyr residue at position 235 of SEQ ID NO: 2 to a Glu. Like the serotonin receptor, this mutation was identified based on the high degree of homology at positions surrounding the -13 position relative to the “CWLP” motif, which is found in many G protein coupled receptors, and the fact that mutations in this region often yielded receptors having increased basal activity (i.e., constitutive activity). Amino acid substitutions that altered the charge of amino acid 235 and surrounding residues were tested to determine whether any of these mutations yielded receptors having alterations in basal activity.

The Tyr235Glu CCR-3 receptor was tested using a transcriptional reporter assay capable of measuring the basal level signaling of the CCR-3 receptor. Cells were cotransfected with a luciferase reporter construct containing a serotonin response element (SRE), an expression vector encoding a chimeric protein called Gq5i (described in detail below), and one of either the wild-type CCR-3 receptor, the Tyr235Glu CCR-3 receptor, or negative control pcDNA1. As shown in Example 2 below, the CCR-3 receptor consistently yielded a decrease in receptor induced basal transcriptional activity.

Identification of Receptors having an Optimized Signal to Noise Ratio

Based on the present invention, one skilled in the art would clearly understand that in order to identify additional receptors having an optimized signal to noise ratio, one could screen for receptors having a decreased level of basal activity or an increased level of ligand-stimulated activity. Some receptors (e.g., wild-type receptors) are naturally silenced, i.e., have a particularly low level of basal activity (i.e., lower than the activity of a negative control). Such naturally occurring silenced receptors are identified by simply comparing the basal activity of the wild-type receptor to that of a negative control. A suitable negative control is, for example, a cell lacking expression of the natural wild-type receptor (e.g., a cell transfected with an empty expression vector, or a cell transfected with a different receptor that has been previously established to be a silenced receptor (preferably both an empty expression vector and a vector including a non-silenced receptor are used in a single experiment as controls)). Alternatively, in order to identify activated receptors one could screen for receptors having an increase in the maximal level of ligand induced signaling.

The skilled artisan will appreciate that novel silenced and activated receptors can be identified using routine screening methods. For example, receptors having silencing mutations may be identified systematically by 1) identifying regions of homology between a particular non-silenced receptor and one or more receptors having alterations in the basal activity of the receptor (e.g., a decrease in the basal activity (silenced receptors) or an increase in the basal activity (constitutively active receptors)); 2) introducing mutations into one or more regions of the non-silenced receptor based on the identified region(s) of homology; and 3) assaying the mutant receptor for a decrease in the basal level of activity. One skilled in the art will appreciate that the mutations can also be introduced by any random mutagenesis procedure standard in the art. A large variety of random mutagenesis kits are in fact commercially available. Once identified, the activity of the receptor may be confirmed, for example, using a mammalian expression system, particularly a yeast expression system.

Applicants demonstrate step 2) by identifying highly conserved regions between the human wild-type serotonin 2A receptor, the rat serotonin 2A receptor, and a number of other constitutively active Class I G protein-coupled receptors. This information was used to target specific residues in the wild-type serotonin 2A receptor for mutation. As described in detail below, a series of targeted point mutations were introduced into the serotonin 2A receptor and the mutant receptors were assayed for decreased basal activity. One mutant receptor, the Tyr323Glu receptor, was indeed silenced, compared to the corresponding wild-type receptor (see Example 1).

It will be appreciated that this method of comparing non-silenced, wild-type receptors and receptors having altered basal activity to identify regions of conservation may be repeated with any family of related receptors with the goal of targeting regions of homology for mutation, as set forth in steps 1) and 2) above. In addition, as noted above, the skilled artisan could easily modify these methods to identify additional activated receptors.

Any standard mutagenesis protocol may be used to generate candidate silenced or activated receptors. As but one example, a receptor may be subcloned into an expression vector (e.g., pcDNA1.1, which ensures high level expression in COS-7 and HEK 293 cells) and confirmed by restriction enzyme and partial DNA sequence analysis. Next, to generate templates for mutagenesis, BW3 13 bacteria are transformed with the expression vector utilizing helper phage to generate a single stranded uracil template for mutagenesis. Each uracil template represents a single receptor and provides sufficient material to generate at least 20 mutant variants of the corresponding receptor. Oligonucleotide primers are then designed to introduce point mutations into the receptor. As noted above, the amino acid alteration(s) to be introduced are selected to optimize the probability of conferring silence or activated activity. Preferably, the oligonucleotides for mutagenesis are designed to introduce a silent restriction site (i.e., one which does not alter the amino acid sequence) in parallel, thus allowing rapid screening of candidate mutant receptor cDNAs. This exemplary technique permits the rapid generation of mutant receptor cDNAs without the need for either PCR or ligations into another plasmid.

Once generated, the candidate mutant receptor cDNAs are transformed into bacteria and grown up as mini-preparations of DNA. Restriction enzyme analysis is used to identify mutant clones, which are then sequenced to confirm introduction of the desired mutation. The identified cDNAs encoding the candidate silenced or activated receptor are then transfected into COS-7 or HEK 293 cell lines. Cells are then split into either 12 or 24 well plates in preparation for step 3).

Step 3) involves assaying the mutant receptors for the desired activity, for example, a decrease in basal activity or an increase in ligand-stimulated activity. Of course, it will be appreciated that the basal or ligand-stimulated activity of a particular receptor can be measured by any assay typically used to measure the basal and/or ligand-stimulated activity of the receptor. Any receptor of therapeutic interest having a known ligand will have such an associated assay. To name but a few, changes in the basal or ligand-stimulated level of second messenger signaling may be assessed to identify silenced or activated receptors, respectively, including, but not limited to, changes in basal levels of cAMP, cGMP, ppGpp, inositol phosphate, or calcium ion. As but one example, ligand-dependent activation of the melanocortin-4 (MC-4) receptor is assayed by measuring the dramatic increase in cAMP formation (Huszar et al., Cell 88:131-141, (1997)). Formation of cAMP is quantified using a radioimmunoassay. The mutant receptors are studied in parallel with wild-type control receptors, cells transfected with an empty expression vector (e.g., pcDNA 1.1 lacking a nucleic acid sequence encoding a receptor), and untransfected cells. In addition, known silenced or activated receptors may be studied in parallel in each assay as positive controls.

These simple principles can easily be applied to identify additional silenced or activated receptors of different types (e.g., nuclear receptors, single transmembrane receptors, or G protein-coupled receptors). Assays used to identify constitutively active receptors could be modified to identify receptors having a decrease, rather than an increase, in basal activity, or to identify receptors having an increase in ligand-stimulated activity. To illustrate this point, the following examples are provided, which are specific to G protein-coupled receptors that have an increase in basal activity (i.e., are constitutively active). As one example, studies that measured increases in intracellular cAMP were carried out to identify constitutively active mutants of the pituitary adenylate cyclase activating polypeptide type I receptor (PAC1) (Cao et al., FEBS Lett., March 10;469(2-3):142-146, (2000)). As another example, the constitutively active mutants of the β2 bradykinin (BK) receptor and the AT1A angiotensin I and II receptors were identified by measuring inositol phosphate production (Marie et al., Mol. Pharmacol. 1:92-101, (1999); Groblewski et al., J. Biol. Chem., 272(3):1822-1826, (1997); Feng et al., Biochemistry, 37(45):15791-15798 (1998)). A constitutively active CCK-BR was also identified by measuring basal inositol phosphate production (Beinborn et al., J. Biol. Chem. 273(23): 14146-14151 (1998)). Mutants of CCK-BR were tested by simply comparing the basal level of inositol phosphate production of a mutant CCK-BR to the basal level inositol phosphate production of the wild-type CCK-BR to determine whether the mutant CCK-BR was constitutively active.

The activity of other types of receptors (e.g., non-G protein-coupled receptors, such as single transmembrane domain receptors and nuclear receptors) can also be measured via the biochemical pathway they induce. For example, binding of the ligand EPO to the EPO receptor activates the JAK2-STAT5 signaling pathway (see, e.g., Yoshimura et al., Curr. Opin. Hematol., 5(3):171-176, 1998). The basal and stimulated levels of JAK2 and STAT5 signaling can easily be assessed by one of ordinary skill in the art, as described in Yoshimura et al., supra, to identify silenced or activated EPO receptors.

As an alternative to measuring molecules in a signaling pathway directly to identify silenced or activated receptors, a reporter assay system may be established in which a response element, responsive to signaling through a particular receptor, is attached to a reporter gene in combination with a transcriptional promoter. Specifically, the expression of the reporter gene is controlled by the activity of the chosen receptor. This method involves the steps of 1) identifying a response element that is sensitive to signaling by a specific receptor polypeptide (e.g., by eliciting an increase or decrease in gene expression upon receptor activation); 2) operably linking the response element and a promoter to a reporter gene; and 3) comparing the basal level reporter activity of a putative silenced receptor to a negative control. A decrease in basal level reporter activity compared to the negative control in the assay indicates the identification of a silenced receptor. A silenced receptor exhibits at least a 25% decrease in basal activity, or at least a 50% decrease in basal activity, or at least a 75% decrease in basal activity, or more than a 100% decrease in basal activity, compared to an appropriate negative control. At the very least, a silenced receptor exhibits a decrease in basal signaling relative to an appropriate negative control that is considered statistically significant using accepted methods of statistical analysis. Alternatively, an increase in the ligand-stimulated activity of a receptor indicates the identification of an activated receptor. An activated receptor exhibits at least a 5% increase, or at least a 10%, 15%, 20%, or 25% increase, or at least a 50%, 60%, or 75% increase, or more than a 100% increase in ligand-stimulated activity, all compared to an appropriate negative control. At the very least, an activated receptor exhibits an increase in ligand-stimulated activity relative to an appropriate negative control that is considered statistically significant using accepted methods of statistical analysis.

It will be appreciated that the receptor can be any receptor identified as a candidate silenced or activated receptor. In addition, one skilled in the art would recognize that the response element used in the present assay can be any response element that is sensitive to signaling through the identified candidate silenced or activated receptor. For example, for reporter assays that are coupled to different G proteins, one would select response elements that are sensitive to signaling through a G protein-coupled receptor. Examples of preferred response elements include a portion of the somatostatin (SMS) promoter (which has included a number of different response elements), the serum response element (SRE), and the cAMP response element (CRE), which are response elements sensitive to G protein-coupled receptor signaling. In particular examples, SMS is activated by coupling of receptors to either Gαq or Gαs; SRE is activated by receptor coupling to Gαq; and CRE is activated by receptor coupling to Gαs and inhibited by coupling to Gαi. Each of these response elements can be employed in a reporter assay to generate a readout for the basal level or ligand-stimulated activity of a specific G protein-coupled receptor.

In addition, a reporter construct for detecting receptor signaling might include a response element that is a promoter sensitive to signaling through a particular receptor. For example, the promoters of genes encoding epidermal growth factor, gastrin, or fos can be operably linked to a reporter gene for detection of G protein-coupled receptor signaling. Another example includes the TPA response element, which is sensitive to phorbol ester induction and activated by receptor coupling to Gαq.

It will be appreciated that a wide variety of reporter constructs can be generated that are sensitive to any of a variety of signaling pathways induced by signaling through a particular receptor (e.g., a second messenger signaling pathway). Accordingly, this assay system may be used to identify other types of silenced or activated receptors, including receptors that are single transmembrane receptors or nuclear receptors, by simply selecting a response element that is sensitive to the particular receptor and positioning the response element upstream of a reporter gene in a reporter construct. For example, the elements AP-1, NF-κb, SRF, MAP kinase, p53, c-jun, TARE can all be positioned upstream of a reporter gene to obtain reporter gene expression. Additional response elements, including promoter elements, can be found in the Stratagene catalog (PathDetect® in Vivo Signal Transduction Pathway cis-Reporting Systems Introduction Manual or PathDetect® in Vivo Signal Transduction Pathway trans-Reporting Systems Introduction Manual, Stratagene, La Jolla, Calif.).

A typical G protein-coupled reporter assay system includes 1) a reporter construct containing a response element that is sensitive to signaling through a specific G protein, and a promoter, operably linked to a reporter gene; preferably in combination with 2) an expression vector containing a promoter operably linked to a nucleic acid encoding a receptor, wherein the receptor is coupled to a G protein or other downstream mediator to which the selected response element is sensitive. The assays used to identify the silenced receptors of the present invention demonstrate use of a specific response element, the serum response element (SRE), which is sensitive to signaling through Gαq (see Tables 1-5).

Several variations of G protein-coupled receptor assays may be used in the present invention. For example, Gαi-mediated decreases in intracellular cAMP can be measured by 1) stimulating cells with forskolin, which causes receptor-independent activation of adenylate cyclase and generates an intracellular pool of cAMP; 2) stimulating the cells with ligand; and 3) measuring the ligand-induced, receptor-dependent Gαi-mediated decrease in the intracellular cAMP pool (e.g., using a radioimmunoassay (e.g, New England Nuclear, Boston, Mass.)).

Alternatively, Gαi coupling can also be measured using a positive assay (i.e., one that yields an increase in activity upon receptor activation), instead of a negative assay (i.e., one that yields a decrease in activity upon receptor activation). This assay provides a more detectable output signal and less interassay variation. In one such assay, a chimeric G protein (Gq5i, Broach and Thomer, Nature 384 (Suppl.):14-16 (1996)) that contains the entire Gαq protein having the five C-terminal amino acids from Gαi attached to the C-terminus of Gαq is used. This chimeric G protein is recognized as Gαi by Gαi coupled receptors, but switches the receptor induced signaling from Gαi to Gαq. This allows Gαi receptor coupling to be detected using a positive assay by use of a Gαq responsive SMS-Luc or SRE-Luc construct (Stratagene, La Jolla, Calif.). SMS and SRE preferably respond to Gαq mediated inositol and calcium production. Moreover, detection can be carried out in the absence of forskolin pre-stimulation of cells. These types of assays are demonstrated in Example 2, below.

Applications

In preferred embodiments, silenced or activated receptors, for example, the Lys323Glu serotonin 2A receptor and the Tyr235Glu CCR-3 receptor, which have increased signal to noise ratios, are used in large scale screening assays to identify receptor specific ligands. In one preferred embodiment, the identified silenced and activated receptors are used as tools for identifying a ligand of a given receptor, including peptide, non-peptide, and small molecule ligands. For example, ligands (e.g., hormones or drugs) that bind to a particular silenced or activated receptor may be identified using a reporter assay system by (1) operably linking a response element, which is sensitive to receptor activation, and a promoter, to a reporter gene to generate a receptor activation sensitive reporter construct; (2) cotransfecting cells with the reporter construct and an expression vector containing nucleic acid encoding the silenced or activated receptor; (3) contacting the cells with a candidate ligand; and 4) assaying for alterations in the basal or ligand-stimulated activity of the reporter construct, an increase or decrease in the ligand-dependent activation of the silenced or activated receptor, compared to ligand-independent signaling, indicating the presence of an agonist or antagonist, respectfully. Ligands that activate or inhibit a particular receptor by increasing or decreasing receptor activity are valuable candidate therapeutic drugs or lead compounds for therapeutics.

Those skilled in the art will appreciate that the administration regimen for a particular pharmaceutical composition can be easily and routinely determined. For example, any ligand identified using the receptors described herein may be combined with a pharmaceutically acceptable carrier and administered to an individual (e.g., a mammal, preferably a farm animal, a zoo animal, a pet, or a human) to treat or prevent disease, or to improve the health of an individual (Remington's Pharmaceutical Sciences, 15^{th} Ed. Easton: Mack Publishing Co. pp. 1405-1412 and 1461-1487 (1975) and The National Formulary XI., 14^{th} Ed. Washington: American Pharmaceutical Association (1975), the contents of which are incorporated herein by reference).

In another embodiment, the silenced or activated receptors of the present invention are used with a panel of reporter gene constructs that are sensitive to different signaling pathways (e.g., SRE-Luc, SMS-Luc, and CRE-Luc) to identify the signaling pathway induced by a particular receptor (e.g., cAMP, inositol phosphate production). This information facilitates and accelerates both the identification of cognate endogenous ligands (i.e., the de-orphaning of a receptor) and the discovery of drugs that act on orphan receptors (e.g., by using the silenced or activated receptors of the invention in a high-throughput drug screening assay). This allows drug screening efforts to be more focused and to be carried out at reduced cost. In addition, no knowledge of the endogenous ligand is needed as a prerequisite for drug screening (as is also true for competitive binding assays).

In a related embodiment, the silenced and activated receptors of the invention are used to identify the G protein to which a particular receptor is coupled in the form of reporter constructs responsive to Gαq, Gαs, or Gαi-mediated signaling. The silenced or activated receptors of the present invention can be used with a panel of reporter constructs that are capable of determining which G protein a particular receptor is coupled to, selected from Gαq, Gαs, and Gαi. The assay system requires (1) a panel of reporter constructs containing a response element sensitive to a particular G protein, selected from Gαq, Gαs, and Gαi (e.g., SMS-Luc, SRE-Luc, and CRE-Luc) and a promoter operably linked to a reporter gene; (2) an expression vector encoding the silenced or activated G protein-coupled receptor; and (3) a cell into which to deliver the components of (1) and (2).

Alternatively, the G protein to which a silenced or activated receptor is coupled may be identified by (1) selecting a silenced or activated G protein-coupled receptor; (2) using an expression vector encoding the selected silenced or activated G protein-coupled receptor in combination with a panel of reporter assays that are capable of detecting coupling to Gαq, Gαs, or Gαi (as described above); and (3) comparing the signal generated by each assay in response to ligand stimulation, an increase in reporter activity in one reporter assay, and not the other two, indicating coupling to the G protein to which the reporter assay is sensitive. In certain cases, a chimeric G protein (for example, the chimeric G protein, Gq5i (Broach and Thorner, supra)), capable of switching the signaling of the receptor to a different pathway than the wild-type receptor may be advantageous or desired. Preferably this signaling pathway generates a positive signal in the reporter assay, as opposed to a negative signal.

Kits

The present invention further provides therapeutic kits containing receptors having an increased signal to noise ratio. In one preferred embodiment, the kit provides nucleic acids encoding silenced or activated receptors, e.g., the Lys323Glu serotonin 2A receptor or the Tyr235Glu CCR-3 receptor. Preferably, the nucleic acid molecule is a vector that contains a promoter operably linked to the nucleic acid encoding the silenced or activated receptor. In another preferred embodiment, the kit provides cells, e.g., eukaryotic cells, preferably mammalian cells, expressing the silenced or activated receptors on the cell surface.

According to the present invention, the kits provide silenced or activated receptors that may be used in large scale screening assays to identify novel ligands for the receptors. In one preferred embodiment, the kits include a first container means containing a nucleic acid encoding a silenced or activated receptor, e.g., a vector in an appropriate buffer solution. In another preferred embodiment, the first container means contains cells expressing the silenced or activated receptors on the cell surface in a media solution that enables survival of the cells during the period of time required for delivery of the kit to the consumer. Also included in the kit may be reagents and solutions required to grow the cells upon arrival in third, fourth, etc. container means. In particularly preferred embodiments, the silenced or activated receptors include the Lys323Glu serotonin 2A receptor or the Tyr235Glu CCR-3 receptor. In yet another preferred embodiment, the kit may provide a first container means containing cells, e.g., competent cells or non-competent cells, and a second container means containing nucleic acids encoding silenced or activated receptors with which to transfect the cells, and a third, fourth, etc. container means containing additional reagents needed to grow the cells and/or transfect the cells with the DNA, in order to use the silenced or activated receptor for research purposes, e.g., for identifying ligands for the receptor.

The container means can be made of glass, plastic, or foil and can be a vial, bottle, pouch, tube, bag, etc. The kit may also contain written instructions, such as procedures for using the vectors to identify receptor ligands, or analytical information, such as the amount of reagent (e.g. moles or mass of nucleic acid or number of cells). The written information may be located on any of the first, second, and/or third etc., container means, and/or a separate sheet included, along with the first, second, and/or third etc., container means, in a fourth container means. The fourth container means may be, e.g., a box or a bag and may contain the first, second, and third container means. It will be appreciated that this kit can be modified to include any reagent for use described above, or known in the art.

All references cited herein are hereby incorporated by reference.

### EXAMPLES

The present invention can be further understood through consideration of the following non-limiting examples.

### Example 1

### Serotonin 2A Receptor

This example demonstrates identification of a serotonin 2A receptor having an increased signal to noise ratio due to a decrease in basal activity.

Generating Mutant Serotonin 2A Receptors

Residues highly conserved between many G protein coupled receptors and the serotonin 2A receptor are illustrated in FIG. 1. Of particular interest was the region surrounding the Cys residue at position 322 of SEQ ID NO: 1 (the position 13 amino acids N-terminal to the “CWLP” motif (-13)), which is conserved between the human and rat serotonin 2A receptors. For example, mutation of the Cys at position 322 to a Lys yields a constitutively active receptor. Based on the high degree of conservation in the region surrounding the -13 position in the human, rat, and other constitutively active receptors (e.g., the 1A adrenergic receptor, the α2C adrenergic receptor, the β2 adrenergic receptor, the cholecystokinin-B receptor, the platelet activating factor receptor, and the thyroid stimulating hormone receptor) and the observation that many of the mutations that induced constitutive activity altered the charge at a particular residue in that region, we chose to generate a human serotonin 2A receptor having point mutations that altered the charge of amino acids surrounding the amino acid at position −13. One of these point mutations was a Lys323Glu mutation in the human serotonin 2A receptor. The mutations were introduced using standard molecular biological techniques and subcloned into the expression vector pcDNA1 (Sambrook et al. supra).

Assaying Mutant Serotonin Receptors for Increased Signal to Noise Ratio

Silencing activity of the Lys323Glu human serotonin 2A receptor was assessed using a luciferase assay (LucLite Luciferase Assay Kit, Packard). The human serotonin 2A receptor is a Gαq coupled receptor. Therefore, we chose to use a reporter construct having an SRE. HEK293 cells were transfected with the reporter construct SRE-Luc and an expression vector containing nucleic acid encoding either the wild-type or the Lys323Glu mutant human serotonin 2A receptor. Basal and ligand-stimulated luciferase activity of the mutant receptor was measured. The ligand used in this assay was serotonin. As a negative control, HEK293 cells were transfected with pcDNA1 (empty vector DNA) and SRE-Luc.

Transfected cells were stimulated as follows. Ligands for the receptor, either serotonin or a non-peptide ligand, were diluted to a desired concentration and added to the transfected cells, which were then incubated for the desired time (standard is overnight) at 37° C., 5% CO₂, although the optimal stimulation time may vary depending on the particular receptor used. The optimal incubation time may be determined systematically by testing a range of incubation times and determining which one yields the highest level of stimulation. For concomitant assessment of two ligands (e.g., ligand induced inhibition of forskolin stimulated CRE activity) each stimulus was prepared at two times the desired final concentration and mixed in equal volumes prior to addition to cells. An assay for luciferase expression was carried out according to the manufacturer's instructions (Packard, Meridin, Conn.).

Results: Serotonin Receptor

As shown below, the Lys323Glu human serotonin 2A receptor consistently demonstrated a decrease in basal signaling. The Lys323Glu human serotonin 2A receptor therefore classifies as a receptor having an optimized signal to noise ratio. The results are as follows.

Table 1 represents the average values for a total of 15 separate experiments for the wild-type, 8 separate experiments for the mutant, and 8 separate experiments for the negative control, shown in FIG. 3, that measure receptor stimulation using a luciferase assay.

TABLE 1

| | Basal | Ligand-stimulated | Stimulated/Basal |
|---|---|---|---|
| Receptor | Activity | Activity | Ratio |
| wild-type serotonin 2A | 17,575 | 107,606 | 6.1 |
| receptor (SRE-Luc) | | | |
| Lys323Glu serotonin | 6,199 | 132,127 | 21.3 |
| 2A receptor (SRE-Luc) | | | |
| pcDNA 1 (SRE-Luc) | 8085 | 9334 | 1.6 |

Table 2 represents the average values for a total of 7 separate experiments shown in FIG. 4 for the wild-type and mutant serotonin receptors, and the negative control, as measured by inositol phosphate production.

TABLE 2

| | Basal | Ligand-stimulated | Stimulated/Basal |
|---|---|---|---|
| Receptor | Activity | Activity | Ratio |
| wild-type serotonin 2A | 6.0 | 27.7 | 4.6 |
| receptor | | | |
| Lys323Glu serotonin 2A | 3.8 | 26.1 | 6.9 |
| receptor | | | |
| pcDNA 1 | 1.9 | N/A | N/A |

Table 3 represents the values shown in Table 2, indicated as percentages.

TABLE 3

| | Basal | Ligand-stimulated | Stimulated/Basal |
|---|---|---|---|
| Receptor | Activity | Activity | Ratio |
| wild-type serotonin 2A | 14.6% | 100% | 6.85 |
| receptor | | | |
| Lys323Glu serotonin 2A | 8.5% | 100% | 11.8 |
| receptor | | | |
| pcDNA 1 | defined | N/A | N/A |
| | as 0% | | |

### Example 2

### The CCR-3 Receptor

This example demonstrates the identification of a CCR-3 receptor having an increased signal to noise ratio due to a decrease in basal activity.

Generating Mutant CCR-3 Receptors

Residues that are highly conserved between many G protein coupled receptors and the CCR-3 receptor are illustrated in FIG. 2. As with the serotonin receptor, the region of particular interest was the region surrounding the Ile residue at position 235 of SEQ ID NO: 2 (13 amino acids N-terminal to the “CWLP” motif (−13)), which is conserved between many G protein coupled receptors.

Based on the high degree of conservation in the region surrounding the −13 position in the CCR-30 receptor and other constitutively active G protein coupled receptors, and the observation that many of the mutations that induced constitutive activity altered the charge at a particular residue in the −13 region, we chose to generate a CCR-3 receptor having point mutations that altered the charge of amino acids surrounding position −13. One of these point mutations was a Tyr235Glu mutation in the CCR-3 receptor. These mutations were introduced using standard molecular biological techniques and subcloned into the expression vector pcDNA1 (Sambrook et al. supra).

Assaying Mutant CCR-3 Receptors for Increased Signal to Noise Ratio

The basal activity of the Tyr235Glu mutant CCR-3 receptor was assessed using a luciferase assay. The CCR-3 receptor is a Gαi coupled receptor. Therefore, we chose to use the SRE-Luc+Gq5i reporter system, described in detail above (Broach and Thorner, supra), which switches the signaling pathway from Gαi to Gαq for reliable positive readout. The luciferase assay was carried out as described above for the serotonin 2A receptor.

Results: CCR-3 Receptor

As shown in FIG. 5 and summarized in Table 4 below, the Tyr235Glu CCR-3 receptor demonstrated approximately a decrease in basal activity. The Tyr235Glu CCR-3 receptor therefore classifies as a receptor having an optimized signal to noise ratio. Table 4 shows the average value for a total of 5 separate experiments for the wild-type, mutant, and negative control vectors.

TABLE 4

| | Basal | Ligand-stimulated | Stimulated/Basal |
|---|---|---|---|
| Receptor | Activity | Activity | Ratio |
| wild-type CCR-3 (SRE- | 33,177 | 157,389 | 4.7 |
| Luc + Gq5i) | | | |
| Lys323Glu CCR-3 | 12,626 | 151,445 | 12 |
| (SRE-Luc + Gq5i) | | | |
| pcDNA 1 (SRE-Luc) | 14,659 | N/A | N/A |

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

1. A silenced receptor having an increased signal to noise ratio compared to a corresponding wild-type receptor, said silenced receptor having decreased basal activity compared to said wild type receptor.

2. The receptor of claim 1, wherein said silenced receptor is a G protein-coupled receptor.

3. The receptor of claim 2, wherein said G protein-coupled receptor is a serotonin receptor.

4. The receptor of claim 3, wherein said serotonin receptor is a serotonin 2A receptor.

5. The receptor of claim 4, wherein said serotonin 2A receptor has a Lys to Glu mutation at position 323 of SEQ ID NO: 1.

6. The receptor of claim 2, wherein said G protein-coupled receptor is a CCR-3 receptor.

7. The receptor of claim 6, wherein said CCR-3 receptor has a Tyr to Glu mutation at position 235 of SEQ ID NO: 2.

8. The receptor of claim 1, wherein said silenced receptor is a nuclear receptor.

9. The receptor of claim 8, wherein said nuclear receptor is a steroid hormone receptor.

10. The receptor of claim 1, wherein said silenced receptor is a single transmembrane receptor.

11. An activated receptor having an increased signal to noise ratio compared to a corresponding wild-type receptor, said activated receptor having increased ligand-stimulated activity compared to said wild-type receptor.

12. The receptor of claim 13, wherein said activated receptor is a G protein-coupled receptor.

13. The receptor of claim 13, wherein said activated receptor is a nuclear receptor.

14. The receptor of claim 13, wherein said activated receptor is a single transmembrane receptor.

15. A kit comprising a silenced receptor having an increased signal to noise ratio compared to a corresponding wild-type receptor, said silenced receptor having a decreased basal activity compared to said wild-type receptor.

16. The kit of claim 17, wherein said silenced receptor is a G protein-coupled receptor.

17. The kit of claim 18, wherein said G protein-coupled receptor is a serotonin receptor.

18. The kit of claim 19, wherein said serotonin receptor is a serotonin 2A receptor.

19. The kit of claim 20, wherein said serotonin 2A receptor has a Lys to Glu mutation at position 323 of SEQ ID NO: 1.

20. The kit of claim 18, wherein said G protein-coupled receptor is a CCR-3 receptor.

21. The kit of claim 22, wherein said CCR-3 receptor has a Tyr to Glu mutation at position 235 of SEQ ID NO: 2.

22. The kit of claim 17, wherein said receptor is a nuclear receptor.

23. The kit of claim 17, wherein said receptor is a single transmembrane receptor.

24. A kit comprising an activated receptor having an increased signal to noise ratio compared to a corresponding wild-type receptor, said activated receptor having an increased ligand-stimulated activity compared to said wild-type receptor.

25. The kit of claim 26, wherein said activated receptor is a G protein-coupled receptor.

26. The kit of claim 26, wherein said activated receptor is a nuclear receptor.

27. The kit of claim 26, wherein said activated receptor is a single transmembrane receptor.

28. A method of using a receptor having an increased signal to noise ratio to identify ligands for the receptor, comprising the steps of:
(a) cotransfecting cells with an expression vector containing a nucleic acid encoding said receptor having an increased signal noise ratio and a receptor activation-sensitive reporter construct, said reporter construct comprising an operably linked response element, which is sensitive to activation by said receptor, promoter, and reporter gene;
(b) contacting the cells with a candidate ligand; and
(c) assaying for alterations in the basal or ligand-stimulated activity of said reporter construct, an increase or decrease in the ligand-dependent activation of said receptor, compared to ligand-independent signaling, indicating the presence of an agonist or antagonist, respectfully.

29. The method of claim 30, wherein said receptor having increased signal to noise ratio is a silenced receptor.

30. The method of claim 30, wherein said receptor having increased signal to noise ratio is an activated receptor.

31. The method of claim 31, wherein said silenced receptor is a G protein-coupled receptor.

32. The method of claim 33, wherein said G protein-coupled receptor is a serotonin receptor.

33. The method of claim 34, wherein said serotonin receptor is a serotonin 2A receptor.

34. The method of claim 35, wherein said serotonin 2A receptor has a Lys to Glu mutation at position 323 of SEQ ID NO: 1.

35. The method of claim 33, wherein said G protein-coupled receptor is a CCR-3 receptor.

36. The method of claim 37, wherein said CCR-3 receptor has a Tyr to Glu mutation at position 235 of SEQ ID NO: 2.

37. The method of claim 31, wherein said silenced receptor is a nuclear receptor.

38. The method of claim 31, wherein said silenced receptor is a single transmembrane receptor.

39. The method of claim 32, wherein said activated receptor is a G protein-coupled receptor.

40. The method of claim 32, wherein said activated receptor is a nuclear receptor.

41. The method of claim 32, wherein said activated receptor is a single transmembrane receptor.
